(19) United States
(12) Patent Application Publication
(10) Pub. No.: US 2026/0187988 A1
(43) Pub. Date: Jul. 2, 2026

(54) SYSTEM AND METHOD FOR ASSISTING MEDICAL ANNOTATION FOR SELF-IMPROVING REPRODUCIBILITY

(71) Applicant: AJOU UNIVERSITY INDUSTRY-ACADEMIC COOPERATION FOUNDATION, SUWON-SI (KR)

(72) Inventors: Jung Won LEE, Seoul (KR); Hyeon-jin CHOI, Gyeonggi-do (KR)

(73) Assignee: AJOU UNIVERSITY INDUSTRY-ACADEMIC COOPERATION FOUNDATION, SUWON-SI (KR)

(21) Appl. No.: 19/373,614

(22) Filed: Oct. 29, 2025

(30) Foreign Application Priority Data
Dec. 26, 2024 (KR) 10-2024-0196427

(51) Int. Cl. G06V 10/778; G06V 10/25; G06V 10/774; G06V 10/776

(52) U.S. Cl. CPC G06V 10/7788; G06V 10/25; G06V 10/774; G06V 10/776; G06V 2201/03

(57) ABSTRACT

A method of assisting a medical annotation for self-improving reproducibility is disclosed. The method includes receiving, from the user terminal, a first data including a medical image and a bounding box information displayed on the medical image; inputting the first data into a pre-trained learning model having a first performance to generate a prediction region information, which represents information on a masking region; receiving, from the user terminal, a second data including the medical image, the bounding box information, and a target region information obtained by modifying the prediction region information; additionally training the learning model based on the second data; evaluating a second performance of the learning model after the additional training is completed; and determining whether a reproducibility criterion under a preset condition is satisfied, and requesting the user terminal to provide additional second data for additional training if the reproducibility criterion is not satisfied.

### CROSS-REFERENCE TO RELATED APPLICATION

The present application claims priority under 35 U.S.C. § 119(a) to Korean patent application number 10-2024-0196427 filed on December 26, 2024, in the Korean Intellectual Property Office, the entire disclosure of which is incorporated by reference herein.

### BACKGROUND

### 1. Technical Field

The present disclosure relates to a system and method for assisting medical annotation (hereinafter, “medical annotation assisting system and method,” which may be used interchangeably) for self-improving reproducibility.

### 2. Related Art

Diagnosis using medical images captured such as X-rays is the most commonly used method in clinical sites. In this regard, model development utilizing medical image data has been actively performed in the field of medical artificial intelligence.

However, medical data is difficult to collect because of privacy protection issues, and it is further difficult to collect since annotation tasks such as image classification, bounding box generation, and mask data generation require expertise.

Mask generation is a task of marking, on a pixel basis, a region of an object to be trained by artificial intelligence, which requires the longest working time and causes high fatigue to a worker. In particular, mask generation for medical data with unclear boundaries is even more difficult. For example, in a chest X-ray image, since pulmonary nodules are difficult to identify, a bounding box is first marked, and then a region of the nodule within the bounding box is identified and marked.

Mask generation for medical data is a very laborious task when performed manually, and it is difficult to generate a large amount of data for developing artificial intelligence models.

In order to solve such problems, the related art has proposed a masking method and system for constructing medical image data for artificial intelligence training. The invention includes a step of receiving medical images, preprocessing the images, inputting the preprocessed data into an artificial intelligence algorithm to obtain a recommended region of interest, and enabling a doctor to generate mask data with reference thereto. The invention introduced artificial intelligence technology to improve mask data generation work of the doctor. However, it is merely a masking work assistance tool that relies entirely on the capability of a pre-trained artificial intelligence, and reproducibility cannot be expected for data generated in different environments.

Reproducibility refers to the ability of an artificial intelligence model to reproduce performance on different datasets, and is a very important performance indicator in the medical field where data exhibits different

characteristics depending on imaging equipment, internal parameters, and imaging environments.

However, achieving high reproducibility in the field of medical artificial intelligence is a difficult problem, and many models with high performance still lack sufficient reproducibility. That is, it is difficult to expect that a model trained only with data owned by a developer will function with high performance in various environments.

### SUMMARY

The technical problem to be solved by the present disclosure is to provide a medical annotation assisting system and method for self-improving reproducibility, which can be customized for user-specific environments by performing user-specific fine-tuning until sufficient reproducibility is achieved.

In order to solve the above-mentioned technical problems, a method of assisting a medical annotation for self-improving reproducibility, according to an embodiment of the present disclosure may be performed by at least one processor of a computing device that communicates with a user terminal, the method including: receiving, from the user terminal, a first data including a medical image and a bounding box information displayed on the medical image; inputting the first data into a pre-trained learning model having a first performance to generate a prediction region information, which represents information on a masking region; receiving, from the user terminal, a second data including the medical image, the bounding box information, and a target region

information obtained by modifying the prediction region information; additionally training the learning model based on the second data; evaluating a second performance of the learning model after the additional training is completed; and determining whether a reproducibility criterion under a preset condition is satisfied, and requesting the user terminal to provide additional second data for additional training if the reproducibility criterion is not satisfied, wherein it may be determined whether the reproducibility criterion is satisfied based on a comparison result between the first performance and the second performance.

According to an embodiment of the present disclosure, the method may further include executing each step with respect to another user terminal different from the user terminal; and storing additionally trained parameters separately for each user.

In an embodiment of the present disclosure, the reproducibility criterion may be that a reproducibility performance error is less than or equal to a preset value, and the reproducibility performance error may be an error between the first performance and the second performance.

In an embodiment of the present disclosure, the reproducibility criterion may be that the second performance is greater than or equal to a preset value, and the second performance may be an Intersection over Union (IoU) index.

In an embodiment of the present disclosure, the additional training may be performed to adjust only parameters of some layers of the learning model.

In order to solve the above-mentioned technical problems, a system for assisting a medical annotation for self-improving reproducibility according to an embodiment of the present disclosure includes a communicator configured to transmit and receive information to and from a user terminal; a memory configured to store a pre-trained learning model having a first performance; and a processor, wherein the processor is configured to receive, from the user terminal, a first data including a medical image and a bounding box information displayed on the medical image, input the first data into the learning model to generate a prediction region information, which represents information on a masking region, receive, from the user terminal, a second data including the medical image, the bounding box information, and a target region information obtained by modifying the prediction region information, additionally train the learning model based on the second data, evaluate a second performance of the learning model after the additional training is completed, and determine whether a reproducibility criterion under a preset condition is satisfied, and request the user terminal to provide additional second data for additional training if the reproducibility criterion is not satisfied, wherein it may be determined whether the reproducibility criterion is satisfied based on a comparison result between the first performance and the second performance.

The present disclosure has an effect of securing individual reproducibility, which can be customized for user-specific environments, by performing user-specific fine-tuning until sufficient reproducibility is achieved.

### BRIEF DESCRIPTION OF THE DRAWINGS

FIG. 1 illustrates a medical annotation assisting system for self-improving reproducibility according to an embodiment of the present disclosure.

FIG. 2 illustrates a part of a configuration of a medical annotation assisting system for self-improving reproducibility according to an embodiment of the present disclosure.

FIG. 3 illustrates a medical annotation assisting system for self-improving reproducibility according to an embodiment of the present disclosure.

FIG. 4 illustrates a part of a configuration of a medical annotation assisting system for self-improving reproducibility according to an embodiment of the present disclosure.

FIG. 5 schematically illustrates a bounding box and a masking process for a medical image.

FIG. 6 illustrates a medical annotation assisting method for self-improving reproducibility according to an embodiment of the present disclosure.

### DETAILED DESCRIPTION OF EMBODIMENT

This invention was made with support from the National Research and Development Program of Korea. The information of the supported project is as follows:

[Assignment Unique Number] 2710033965

[Name of the Ministry] Korea Ministry of Science and ICT

[Name of the Assignment Managing (Professional) Organization]

### Institute of Information & Communications Technology Planning & Evaluation IITP

[Research Project Title] University ICT Research Center (ITRC)

[Assignment Title] Development of Intelligent Medical Imaging Diagnosis Solution

[Name of the Organization Performing the Assignment] Ajou University Industry-Academic Cooperation Foundation

[Research Period] 2020.07.01 ∼ 2027.12.31

The present disclosure may undergo various modifications and may have various embodiments, and specific embodiments are illustrated in the drawings and will be described in detail. However, it is not intended to limit the present disclosure to the specific embodiments, and it should be understood that the present disclosure encompasses all modifications, equivalents, and alternatives within the spirit and scope of the present disclosure.

In describing the present disclosure, a detailed description of related known techniques will be omitted when it is judged that the subject matter of the present disclosure may obscure.

Hereinafter, embodiments of the present disclosure will be described in detail with reference to the accompanying drawings.

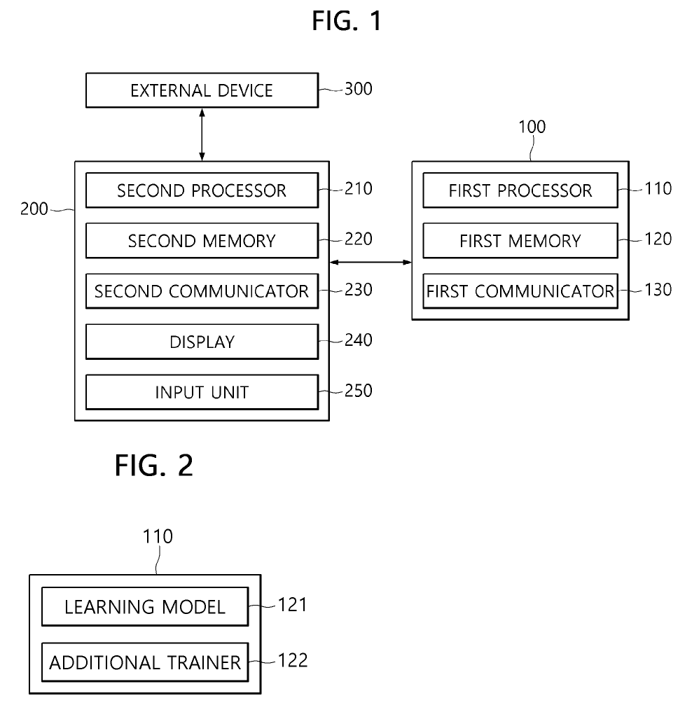

FIG. 1 illustrates a medical annotation assisting system for self-improving reproducibility according to an embodiment of the present disclosure (hereinafter, also simply referred to as “system”).

Referring to FIG. 1, the system includes a server 100 and a user terminal 200.

The server 100 includes a first processor 110, a first memory 120, and a first communicator 130.

The first processor 110 is configured to process information related to annotation assistance and may include at least one processor.

The first processor 110 may include at least one of a central processing unit (CPU), a graphics processing unit (GPU), a microprocessor, and an artificial intelligence (AI) dedicated processor, and the type and number of processors are not limited as long as they perform functions of the present disclosure.

The first memory 120 may store a program including data and executable instructions that can be read or written by the first processor 110.

The first memory 120 includes a non-volatile memory capable of retaining data (information) regardless of whether power is supplied, and a volatile memory in which data to be processed by the processor is loaded and which loses the data when power is not supplied. The non-volatile memory may include a flash memory, a hard disk drive (HDD), a solid state drive (SSD), and a read-only memory (ROM), and the like, and the volatile memory may include a buffer and a random access memory (RAM), and the like.

The first communicator 130 may operate under the control of the first processor 110 and transmit and receive information to and from the user terminal 200.

The first communicator 130 may communicate using at least one of a wired/wireless LAN, Wi-Fi, Bluetooth, Zigbee, Infrared Data Association (IrDA), Near Field Communication (NFC), Wireless Broadband Internet (WiBro), Shared Wireless Access Protocol (SQAP), and an RF communication method, but the communication method is not limited to the above embodiment.

Since the first memory 120 and the first communicator 130 are controlled by the first processor 110, operations of the first memory 120 or the first communicator 130 may be described or understood as being performed by the first processor 110.

The user terminal 200 includes a second processor 210, a second memory 220, a second communicator 230, a display 240, and an input unit 250.

The second processor 210 is configured to generate or transmit information related to information processing for annotation assistance and may include at least one processor.

The second processor 210 may include at least one of a central processing unit (CPU), a graphics processing unit (GPU), a microprocessor, and an artificial intelligence (AI) dedicated processor, and the type and number of processors are not limited as long as they perform functions of the present disclosure.

The second memory 220 may store a program including data and executable instructions that can be read or written by the second processor 210.

The second memory 220 includes a non-volatile memory capable of retaining data regardless of whether power is supplied, and a volatile memory in which data to be processed by the processor is loaded and which loses the data

when power is not supplied. The non-volatile memory may include a flash memory, a hard disk drive (HDD), a solid state drive (SSD), a read-only memory (ROM), and the like, and the volatile memory may include a buffer, a random access memory (RAM), and the like.

The second communicator 230 may operate under the control of the second processor 210 and transmit and receive information to and from the server 100 and an external device 300.

The external device 300 may generate a medical image by an imaging device such as an X-ray device. The generated medical image may be transmitted to the user terminal 200.

The second communicator 230 may communicate using at least one of a wired/wireless LAN, Wi-Fi, Bluetooth, Zigbee, Infrared Data Association (IrDA), Near Field Communication (NFC), Wireless Broadband Internet (WiBro), Shared Wireless Access Protocol (SQAP), and an RF communication method, but the communication method is not limited to the above embodiment.

The display 240 visually displays medical image information.

The display 240 may include a liquid crystal display (LCD), a light emitting diode (LED) display, an organic light emitting diode (OLED) display, a micro electro mechanical systems (MEMS) display, and an electronic paper display. The display 240 may also be implemented as a touch screen.

The input unit 250 may include input interfaces such as a keyboard and a mouse. The input unit 250 may include an input function of the touch screen.

Since the second memory 220, the second communicator 230, the display 240, and the input unit 250 are controlled by the second processor 210, operations of the second memory 220, the second communicator 230, the display 240, or the input unit 250 may be described or understood as being performed by the second processor 210.

FIG. 2 illustrates the first memory 120 in detail.

The first memory 120 includes a learning model 121 and an additional trainer 122.

The learning model 121 may include a deep neural network (DNN) structure. An architecture of the learning model 121 may employ any of a convolutional neural network (CNN), a recurrent neural network (RNN), a restricted Boltzmann machine (RBM), a deep belief network (DBN), and a Q network, but is not necessarily limited to these examples.

The learning model 121 may be a pre-trained model with a training dataset that uses a medical image and bounding box information as input data and information on a masking region of an object of interest as output data. The masking region may be segmentation information obtained by distinguishing, for example, a pulmonary nodule region from other regions when the medical image is a chest X-ray (CXR) image.

The additional trainer 122 fine-tunes the pre-trained learning model 121 based on additional teacher data.

The fine-tuning may freeze parameters of some layers of the learning model 121 and adjust only parameters of other layers. For example, fine- tuning may adjust, through training, only a parameter of a final layer among a plurality of layers in the neural network structure. For example, the final layer may be a classifier.

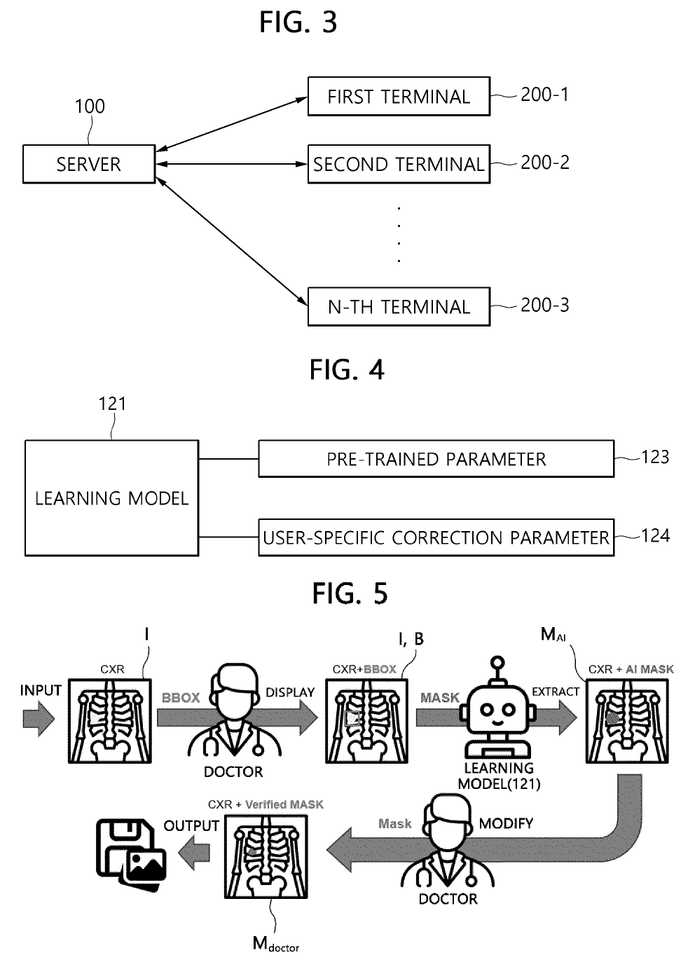

FIG. 3 illustrates a medical annotation assisting system for self-improving reproducibility according to an embodiment of the present disclosure.

Referring to FIG. 3, the server 100 may communicate with a plurality of user terminals to implement a medical annotation assisting method for self-improving reproducibility.

The server 100 may store respective learning parameters fine-tuned based on respective masking information fed back by each of n different users using a first terminal 200-1, a second terminal 200-2, …, and an n-th terminal 200-n.

FIG. 4 illustrates parameters stored in relation to the learning model 121.

A pre-trained parameter 123 is a parameter stored as a result of pre-training of the learning model 121. The pre-trained parameter 123 may be a single type of parameter and may be commonly applied to all users.

A user-specific correction parameter 124 may be a parameter fine-tuned for each user and may be stored separately for each user.

The pre-trained parameter 123 and the user-specific correction parameter 124 may be used individually for each user as a user-customized learning model 121 together with a structure of the learning model 121.

FIG. 5 schematically illustrates a bounding box and a masking process for a medical image.

Referring to FIG. 5, the user terminal 200 receives a medical image (I) from an external device 300.

The user may be a doctor as a medical expert.

The user checks a medical image through the display 240 of the user terminal 200 and inputs a bounding box for the medical image through the input unit 250.

The bounding box may refer to a region of interest (ROI) that limits a part of the medical image, for example, to a rectangular area.

First data (I, B) includes a medical image and bounding box information.

The first data (I, B) may be transmitted from the user terminal 200 to the server 100.

The learning model 121 of the server 100 generates a prediction region (M_{AI}), which is information on masking inferred based on the first data (I, B).

The server 100 transmits the prediction region (M_{AI}) to the user terminal 200.

The user may verify or modify the prediction region (M_{AI}) displayed on the medical image (I) through the display 240 to generate a target region (M_{doctor}), which is modified masking information.

The user terminal 200 determines the target region (M_{doctor}) information obtained by modifying the prediction region (M_{AI}), according to user input information.

The modified target region (M_{doctor}) information may be used as basic diagnostic information for the user and, at the same time, may be provided to the server 100 to be utilized as a training dataset for fine-tuning.

Hereinafter, a detailed description of fine-tuning and reproducibility will be provided through a medical annotation assisting method for self-improving reproducibility.

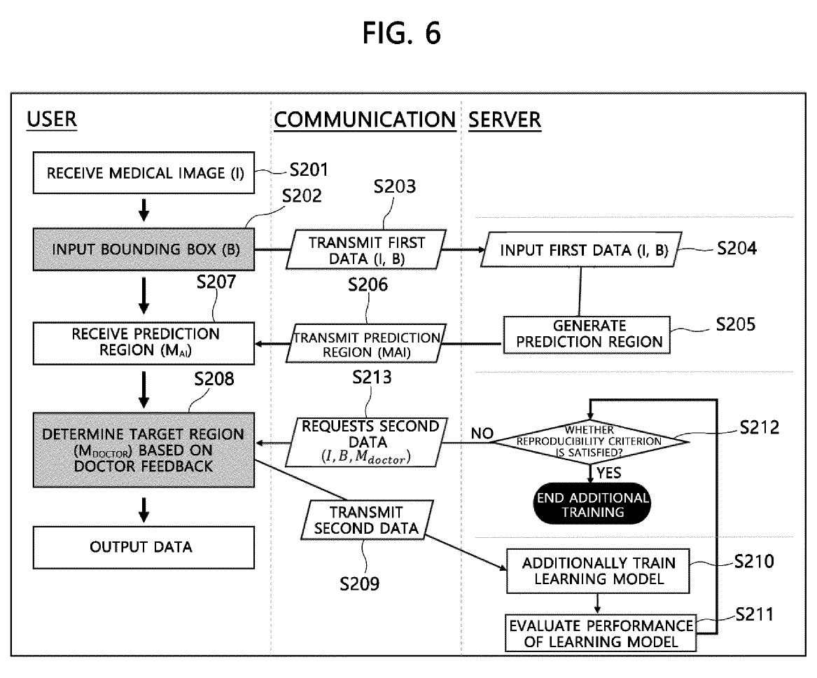

FIG. 6 illustrates a medical annotation assisting method for self-improving reproducibility according to an embodiment of the present disclosure. The medical annotation assisting method for self-improving reproducibility according to an embodiment of the present disclosure may be performed by the medical annotation assisting system for self-improving reproducibility according to an embodiment of the present disclosure.

Referring to FIG. 6, at operation S201, the user terminal 200 receives a medical image.

At operation S202, the user checks the medical image through the display 240 and inputs bounding box information through the input unit 250.

At operation S203, the user terminal 200 transmits first data including the medical image and the bounding box information to the server 100 through the second communicator 230.

At operation S204, the server 100 inputs the received first data into the learning model 121.

At operation S205, the learning model 121 generates prediction region information as output data by using the first data as input data.

The prediction region information includes information on a masking region.

At operation S206, the server 100 transmits the prediction region information to the user terminal 200 through the first communicator 130.

At operation S207, the user terminal 200 receives the prediction region information.

At operation S208, the user inputs a target region obtained by modifying at least a part of the prediction region through the input unit 250. The user terminal 200 determines the target region according to the input data.

Second data includes the medical image, the bounding box information, and the target region information.

At operation S209, the user terminal 200 transmits the second data to the server 100.

The user terminal 200 may transmit the second data to the server 100 only when there is a data request.

At operation S210, an additional trainer 122 of the server 100 additionally trains the learning model 121 by using the received second data as additional training data.

Additional training may be conducted after sufficient additional training data is accumulated. Until sufficient additional training data is accumulated, operations S201 to S209 may be performed a plurality of times on different medical images.

Parameters adjusted through additional training may be stored separately for each user.

At operation S211, the server 100 evaluates performance of the learning model 121 that has completed the additional training.

The performance evaluation may be made based on a degree of overlap between the prediction region and a ground-truth region. For example, an index of the performance evaluation may be an Intersection over Union (IoU) or a Dice score.

At operation S212, the server 100 determines whether the additionally trained learning model 121 satisfies a preset reproducibility criterion. If the reproducibility criterion is satisfied, the server 100 ends the additional training and stores a user-specific correction parameter 124 for each user. If the reproducibility criterion is not satisfied, the server 100 proceeds to operation S213.

The reproducibility criterion may be whether a reproducibility performance error is less than or equal to a preset value.

The reproducibility performance error may refer to an error between a first performance and a second performance.

The first performance refers to performance of the learning model 121 based on parameters before additional training, and the second performance may refer to performance of the learning model 121 based on parameters after additional training.

For the reproducibility performance error, an error of about 1% may be recommended in sensitive fields, while an error of about 10% may be allowed in general fields.

In the case of low-risk artificial intelligence, an error of about 6.8% may also be allowed.

In an embodiment of the present disclosure, satisfaction of the reproducibility criterion may be set when the reproducibility performance error is 5% or less.

The reproducibility criterion may also be set as a numerical value related to performance. For example, when the performance evaluation is based on the IoU index, if the first performance is 90%, the second performance may be required to be 80% or more. In this case, when described as a criterion for a reproducibility performance error, it may be that the reproducibility performance error is 10% or less.

At operation S213, the server 100 additionally requests second data from the user terminal 200. Here, the second data refers to data including a bounding box and a target region generated from a new medical image rather than from a pre-trained medical image.

The present disclosure can replace annotation work of the doctor through inference by the learning model 121, thereby contributing to reducing workload and time cost of the doctor.

In addition, the present disclosure allows inference results of the learning model 121, which may vary depending on imaging equipment or imaging environments of respective clinical sites, to be adjusted. That is, by fine-tuning the learning model 121 with additional training data reflecting the clinical site environments, an effect of providing inference by the learning model 121 adapted to each user can be achieved.

In addition, the present disclosure allows parameters of the learning model 121 to be fine-tuned until a preset reproducibility criterion is satisfied, thereby achieving an effect of enabling the learning model 121 to attain high reproducibility in each medical site.

The terminology used herein is for the purpose of describing particular embodiments only and is not intended to be limiting of the present disclosure. As used herein, the terms “include” and “have” specify the presence of stated features, numbers, steps, operations, elements, components, or combinations thereof, but do not preclude the presence or addition of one or more other features, numbers, steps, operations, elements, components, or combinations thereof.

What is claimed is:

1. A method of assisting a medical annotation for self-improving reproducibility, performed by at least one processor of a computing device that communicates with a user terminal, the method comprising:
receiving, from the user terminal, a first data including a medical image and a bounding box information displayed on the medical image;
inputting the first data into a pre-trained learning model having a first performance to generate a prediction region information, which represents information on a masking region;
receiving, from the user terminal, a second data including the medical image, the bounding box information, and a target region information obtained by modifying the prediction region information;
additionally training the learning model based on the second data;
evaluating a second performance of the learning model after the additional training is completed; and
determining whether a reproducibility criterion under a preset condition is satisfied, and requesting the user terminal to provide additional second data for additional training if the reproducibility criterion is not satisfied,
wherein it is determined whether the reproducibility criterion is satisfied based on a comparison result between the first performance and the second performance.

2. The method of claim 1, further comprising:
executing each step of claim 1 with respect to another user terminal different from the user terminal; and
storing additionally trained parameters separately for each user.

3. The method of claim 1, wherein the reproducibility criterion is that a reproducibility performance error is less than or equal to a preset value, and
the reproducibility performance error is an error between the first performance and the second performance.

4. The method of claim 1, wherein the reproducibility criterion is that the second performance is greater than or equal to a preset value, and
the second performance is measured by an Intersection over Union (IoU) index.

5. The method of claim 1, wherein the additional training is performed to adjust only parameters of some layers of the learning model.

6. A system for assisting a medical annotation for self-improving reproducibility, comprising:
a communicator configured to transmit and receive information to and from a user terminal;
a memory configured to store a pre-trained learning model having a first performance; and
a processor,
wherein the processor is configured to:
receive, from the user terminal, a first data including a medical image and a bounding box information displayed on the medical image,
input the first data into the learning model to generate a prediction region information, which represents information on a masking region,
receive, from the user terminal, a second data including the medical image, the bounding box information, and a target region information obtained by modifying the prediction region information,
additionally train the learning model based on the second data,
evaluate a second performance of the learning model after the additional training is completed, and
determine whether a reproducibility criterion under a preset condition is satisfied, and request the user terminal to provide additional second data for additional training if the reproducibility criterion is not satisfied,
wherein it is determined whether the reproducibility criterion is satisfied based on a comparison result between the first performance and the second performance.
